(19) United States
(12) Patent Application Publication
(10) Pub. No.: US 2025/0236919 A1
(43) Pub. Date: Jul. 24, 2025

(54) DNA SEPARATION

(71) Applicant: GENOMIC LABS LTD, Bristol (GB)

(72) Inventors: Daniel Rogerson, Bristol (GB); Clio ANDREAE, Bristol (GB); Sarah PALMER, Bristol (GB); Josh DYER, Bristol (GB)

(21) Appl. No.: 18/853,534

(22) PCT Filed: Apr. 4, 2023
(86) PCT No.: PCT/GB2023/050894

§ 371 Date: Oct. 2, 2024

(30) Foreign Application Priority Data
Apr. 4, 2022 (GB) 2204912.6

(51) Int. Cl. C12Q 1/689; C12N 15/10; C12Q 1/6806

(52) U.S. Cl. CPC C12Q 1/689; C12N 15/1013; C12Q 1/6806; C12Q 2600/156

(57) ABSTRACT

A method of selectively binding DNA of bacterial origin comprising the step of contacting a sample solution containing DNA of bacterial origin with a protein comprising the TgMcrBΔ185N-terminal domain of Thermococcus gammatolerans McrB protein or a derivative thereof which has been immobilised into a substrate whenever the solution further contains DNA of eukaryotic origin at a concentration of more than 1-100 times that of the DNA of bacterial origin.

### FIELD OF THE INVENTION

The invention relates to products and methods for the extraction of prokaryotic DNA from samples containing far greater levels of eukaryotic DNA. For example, it relates to the extraction of the DNA of pathogenic bacteria from clinical samples containing overwhelming levels of eukaryotic host DNA.

### BACKGROUND TO THE INVENTION

There is an incompletely-met need for improvements in extracting prokaryotic DNA from clinical samples (i.e. body fluids, swabs, faecal samples, blood samples, etc.) In many applications it is preferred that the extraction of prokaryotic DNA takes place rapidly so that subsequent assays, which may be vital to guide treatment, can be undertaken and the treatment started without delay. For example, in cases of sepsis shortening the time to diagnosis allows treatment to start sooner when it is more likely to be successful. It is also preferred that prokaryotic DNA be extracted in a non-sequence specific manner. This is especially important where the putative sequence is not known or for example when the prokaryotic DNA that has been extracted at the level low concentrations and using a simple reproducible method.

Because bacteria DNA may form only a small proportion of a clinical sample, it is often difficult to extract it without significant contamination from the host DNA.

Various existing methods, reagents and kits currently exist. A large proportion of kits are based on differential lysis methods or enzymatic digestion of host DNA. Commercially available using such principles include HostZERO™ microbial DNA kit (Zymo Research), MolYsis™ complete 5 kit (Molzym GmbH & Co. KG) and QIAamp™ DNA microbiome kit (Qiagen). Alternative approaches include using a combination of differential lysis and subsequent capture of human host DNA via methylated CpG capture technology (as used in the NEBNext® Microbiome DNA Enrichment kit (New England Biolabs). Comparative studies comparing the efficiency of MolYsis™ Complete 5, Zymo HostZERO™ and NEBNext® microbiome kits found a lower limit of detection for microbial DNA to be equivalent to 103 colony forming units (CFU, essentially viable bacteria) per 1 ml sample for sufficient genome coverage to enable successful genome sequencing in particular sequencing by Next Generation Sequencing (NGS) technology. This is a problem when using NGS in order to profile a suspected sepsis by whole genome sequencing for antimicrobial drug resistance gene detection, or other characterization because blood samples from sepsis patients often contain as few as 10 CFUs per 10 ml of blood. Because the speed in which sepsis is identified and treatment started has a large influence on clinical outcome, methods of identifying sepsis which do not involve time-consuming culture of microbial organisms in order to increase viable cell counts are advantageous.

Eukaryotic and prokaryotic DNA are often differently methylated. This creates an opportunity to differentially select DNA type from the other by targeting commonly occurring methylation motifs, rather than targeting a specific sequence of residues (as in PCR-based methods).

Exploration of DNA modification for DNA enrichment using magnetic beads is currently available commercially and involves binding to methylated CpG motifs in eukaryotic host DNA. This is the basis of the NEBNext® microbiome DNA enrichment kit. The present invention is based on the opposite approach-it exploits an ability of certain proteins to bind modifications that are common in bacterial DNA but rare in eukaryotic DNA.

Restriction enzymes (non-progressive endonucleases) are available that bind to prokaryotic—specific methylation patterns and cleave the nucleic acid. For example, Barnes et al. (PLOS One. 2014; 9(10): e109061) discloses repurposed restriction endonuclease DpnI immobilised on magnetic beads to achieve extraction of bacterial genomes containing the DpnI Gm6 ATC motif common in the genomic sequence of many, but not all, bacteria. U.S. Pat. Nos. 8,927,218B2 and 10,190,113BR (Forsyth) relate to the non-progressive endonucleases modified to bind to, but not leave, a target nucleic acid in order to separate DNA from different environmental or clinical sources. Specifically binding of N4-methylcytosine and N6-methyladenosine is disclosed.

The present invention relates to the use of DNA-binding proteins based on a protein occurring in an archaea extremophile, said to be a repurposed protein capable of selectively binding to bacterial-specific DNA methylation patterns.

Thermococcus gammatolerans is a thermophilic archaeal species that encodes a unique protein known as McrB which comprises a DNA-binding domain that is specific for bacterial m6A DNA (Hosford et al. 2020, J. Biol Chem 295(3):743-756). McrB is part of a two component modification-dependent restriction system that leaves selectively DNA-containing methylated cytosines. MerB occurs in a number of species including Escherichia coli. However the binding domains of McrB are poorly conserved suggesting a diversity in mechanism of DNA-recognition.

Horsford et al. (2020) identified a region of the Thermococcus gammatolerans McrB N-terminal domain (known as TgMcrBΔ185) which binds preferentially to DNA showing m6A methylation.

### SUMMARY OF THE INVENTION

The present invention is based on the use of proteins comprising the TgMcrBΔ185 domain (or homologues thereof) for preferentially capturing bacterial DNA from a mixture containing bacterial DNA and eukaryotic DNA wherein the Eukaryotic DNA is present in overwhelming amounts. It is based not only on the discovery of the useful actuality of TgMcrBΔ185, but also on the surprising discovery that TgMcrBΔ185 has superior binding and selectivity for bacterial DNA and can therefore be used to separate of bacterial DNA from eukaryotic host DNA, wherein the bacterial DNA is present in low concentrations in a mixture comprising overwhelming amounts of eukaryotic DNA.

According to a first aspect of the invention, there is provided a method of selectively binding DNA of bacterial origin comprising the step of:
i) contacting a sample solution containing DNA of bacterial origin with a protein comprising the TgMcrBA185 N-terminal domain of Thermococcus gammatolerans McrB protein or a derivative thereof, which has been immobilised into a substrate whenever the solution further contains DNA of eukaryotic origin at a concentration of more than 10 times (for example at least 100 times or at least 1000 times) that of the DNA of bacterial origin.

According to a second aspect of the invention there is provided a reagent comprising:
a protein comprising the TgMcrBΔ185 N-terminal domain of Thermococcus gammatolerans or a derivative thereof which has been immobilised onto a substrate and which is capable of binding DNA of bacterial origin in preference to DNA of eukaryotic origin.

According to a third aspect of the invention, there is provided a kit comprising a reagent according to any of claims 11 to 13 together with one or more further components selected from:
1. a wash solution for washing the substrate of any DNA of eukaryotic origin which may have bound non-specifically or weakly to the substrate whilst selectively retaining any DNA of bacterial origin, and
2. an elution solution for eluting DNA of bacterial origin from the substrate.

### BRIEF DESCRIPTION OF DRAWINGS

FIG. 1 shows the results of an ELISA binding assay showing TgMcrBΔ185NHis6 binding to m6A methylated DNA.

FIG. 2 shows the result of binding assay using TgMcrBΔ185 in a magnetic bead format.

FIG. 3 shows a binding assay for Escherichia coli DNA.

### DETAILED DESCRIPTION

The present invention is based on the use of proteins comprising the TgMcrBΔ185 domain (or homologues or derivatives thereof) for preferentially capturing bacterial DNA from a mixture containing bacterial DNA and eukaryotic DNA wherein the eukaryotic DNA is present in overwhelming amounts. It is based not only on the discovery of the useful actuality of TgMcrBΔ185, but also on the surprising discovery that TgMcrBΔ185 has superior binding and selectivity for bacterial DNA and can therefore be used to separate of bacterial DNA from eukaryotic host DNA wherein the bacterial DNA is present in low concentrations in a mixture comprising overwhelming amounts of eukaryotic DNA.

According to a first aspect of the invention there is provided a method of selectively binding DNA of bacterial origin comprising the step of:
ii) contacting a sample solution containing DNA of bacterial origin with a protein comprising the TgMcrBΔ185 N-terminal domain of Thermococcus gammatolerans McrB protein or a derivative thereof, which has been immobilised into a substrate whenever the solution further contains DNA of eukaryotic origin at a concentration of more than [X] times that of the DNA of bacterial origin.

According to a second aspect of the invention there is provided a reagent comprising:
a protein comprising the TgMcrBΔ185 N-terminal domain of Thermococcus gammatolerans or a derivative thereof which has been immobilised into a substrate and which is capable of binding DNA of bacterial origin in preference to DNA of eukaryotic origin.

According to a third aspect of the invention there is provided a kit comprising a reagent according to any of claims 11 to 13 together with one or more further components selected from:
1. a wash solution for washing the substrate of any DNA of eukaryotic origin which may have bound non-specifically or weakly to the substrate whilst selectively retaining any DNA of bacterial origin, and
2. an elution solution for eluting DNA of bacterial origin from the substrate.

### Sample Solution

According to all aspects of the invention, the sample solution may be any suitable sample, for example a sample containing bacterial DNA or a sample which is under investigation because it possibly contains bacterial DNA. Preferably, the sample is a clinical or veterinary sample, for example a clinical sample previously obtained for an individual having a bacterial infection or an individual suspected of having a bacterial infection. For example, the individual may have sepsis or a blood stream infection, or be suspected of having sepsis or a bloodstream infection. The individual may have an infection with a bacteria which has, or is suspected of having, genetic resistance to one or more antimicrobial agents. The invention also encompasses non clinical samples for example environmental samples, water samples (for example cooling water samples, wastewater samples, drinking water samples, industrial process water samples), food or drink samples (for example samples taken from food or drink suspected of containing one or more food poisoning organisms or spoilage organisms). Samples of the invention include blood samples (and derivatives therefore such as plasma and serum samples), sputum or pus samples or samples derived from swabs, dressings or any body tissue, body fluid or secretion. According to some embodiments, the methods of the invention include the step of obtaining, and optionally processing the sample. According to other embodiments the method of the invention is carried out on a sample previously obtained prior to the method of the invention.

According to certain embodiments the sample solution comprises DNA of eukaryotic origin (for example, if the sample is a clinical or veterinary sample the DNA of eukaryotic origin may comprise DNA from the host). In certain embodiments the ratio of DNA of bacterial origin versus DNA of eukaryotic origin is at least 1:1, 1:10, at least 1:100, at least 1:1000, at least 1:10000, or at least 1:100000 (as calculated on a weight for weight basis).

### DNA of Bacterial Origin

According to all aspects of the invention, the DNA of bacterial origin may be from any bacteria. According to certain embodiments it may be from a pathogenic bacterial species or strain that is to say from a species or strain which is pathogenic to humans, non-human animals or plants, most preferably pathogenic to humans. According to certain preferred embodiments, the pathogenic organism is selected from the group consisting of Enterococcus spp, Staphylococcus spp, Klebsiella spp, Acinetobacter spp, Pseudomonas spp, and Enterobacter spp, most preferably from the group consisting of Enterococcus faecium, Staphylococcus aureus, Klebsiella pneumoniae, Acinetobacter baumannii, Pseudomonas aeruginosa, and Enterobacter spp. DNA of bacterial origin preferably has methylation patterns typical for bacterial DNA, in particular it has m6A methylation (i.e, the presence of N6-methyladenosine). Preferably, at least 10% (for example, at least 20, 30, 40 50, 60 or 70%) of adenosine bases are methylated. DNA of bacterial origin may optionally be DNA from one or more of the following origins—Pseudomonas aeruginosa, Actineobacter baumanni, Staphylococcus aureus MSSA and MRSA, Enterobacter cloacae, Enterococcus faecalis and Klebsiella pneumonia.

### Protein Comprising the TgMcrBΔ185 N-Terminal Domain of Thermococcus gammatolerans

The amino acid sequence of wild-type TgMcrBΔ185 is show below:

(SEQ ID NO.: 1)
MENQLFIIGIGTGTDEYENFEETILKGVKRNELEGQIGPDILDNCCSDV
CYFWGRSKETIYEKKIDKGDMVLFYVGKRISRNKVDLNQETAVYLGIIC
ETVEISENDVSFLNDFWRKGENFRFLMFFKKKPEKLHHSINEINSKLGY
NPDYFPIAGYVKPERMSGVYDILKNILKKRGILKESDS

Those residues which are bold and underlined (Trp53, Trp115 and Phe121) are aromatic cage residues thought to be essential for m6A binding. Those single underlined and bolded (Tyr61 and Asn82) are involved in DNA binding.

### Derivatives

The scope of the invention relates to TgMcrBΔ185 and also to derivatives of TgMcrBΔ185. In essence, a derivative of TgMcrBΔ185 is to be understood as a protein having at least partial sequence homology with TgMcrBΔ185 and sharing the activity of TgMcrBΔ185, but which differs in either its primary sequence (by substitution, deletion or addition or combinations thereof) or by the addition or removal of chemical moieties (or both).

### Sequence Variants

According to certain embodiments, derivatives of TgMerBΔ185 comprise a sequence which has at least 50, 60, 70, 80, 85, 90, or 95% sequence identity over regions of at least 50 or at least 100 contiguous residues with native TgMcrBA185. According to certain embodiments such derivatives of TgMcrBΔ185 (which may be termed “sequence variants”) may be, or may not be based on homologues of TgMcrBΔ185 in other species.

According to certain embodiments, TgMcrBΔ185 derivatives may comprise residue substitutions, these may be conservative substitutions or non-conservative substitutions. According to certain embodiments, derivatives of TgMcrBΔ185 may be sequence variants in which additional residues may be added, preferable at one or more termini. Such derivatives include derivatives of the engineered with N or C-terminal His-tagged proteins, addition of sortase tagging for alternative to biotinylation, engineered proteins created via directed evolution (to cover generation of large scale mutant libraries for screening purposes).

According to certain embodiments TgMcrBΔ185 derivatives will preferably retain the native residues identified above as forming the “aromatic cage” and sufficient other residues to ensure a correct positioning of those residues relative to each other in the folded protein. According to certain embodiments TgMcrBΔ185 derivatives will preferably have amino acid residues having aromatic side chains at the positions identified above as forming the “aromatic cage” and sufficient other residues to ensure a correct positioning of those residues relative to each other in the folded protein.

According to certain embodiments, derivatives of TgMcrBΔ185 include TgMcrBΔ185 (or sequence variants thereof) which have been substituted with one or more chemical moiety. For example they may be lipidated, acetylated, PEGylated or conjugated to a linked or other molecule. Preferably, such substitution does not materially change the folded configuration of the TgMcrBΔ185 derivative nor its specificity, but substitution may increase the derivatives stability, longevity, ease of manufacture or ease of purification. Preferably, the chemical moiety contributes no more than 100% to the total molecular weight of the resultant molecule.

According to certain embodiments, derivatives of TgMcrBΔ185 comprise a core peptide sequence corresponding to at least 150 contiguous residues beginning from position 1 to 20 of the sequence recited above, wherein no more than 10 residues are substituted or deleted and no more than 10 residues are added to the core peptide sequence and wherein positions 53, 115 and 121 (using the numbering above) are amino acid residues have aromatic side chains (preferably, Trp or Phe, most preferably Trp at positions 53 and 115 and Phe at position 121). Such a derivative may optionally comprise further portions of the sequence above and may optionally include additional peptide sequence appended to either terminal as discussed above. Such a derivative may be optionally linked to further chemical moieties as discussed above.

Preferably, a derivative of the invention comprises between 50 and 250 amino acid residues, more preferably between 150 and 210 amino acid residues. Preferably it has a molecular weight of between 15 and 30 KDa, for example between 18 and 25 KDa.

### Substrates

According to all aspects of the invention, the substrate is a solid material onto which a protein comprising the TgMcrBΔ185 N-terminal domain of Thermococcus gammatolerans McrB protein or a derivative thereof is immobilised. Substrates according to the invention include magnetic substrates (such as magnetic beads or micro participles), glass or silicon substrates (for example nucleic acid microarrays—“chips”) and also microbubbles. According to certain embodiments the substrate may be provided in a column (a hollow tube) or on a surface of laboratory equipment. According to certain embodiments it may be provided in a microfluidic device. The protein comprising the TgMcrBΔ185 N-terminal domain of Thermococcus gammatolerans McrB protein or a derivative thereof may be attached to the substrate directly any a covalent linkage or it may be bound to the substrate via one or more linkers. Linkers include polyethylene glycol linkers. A protein comprising the TgMcrBΔ185 N-terminal domain of Thermococcus gammatolerans McrB protein or a derivative thereof may optionally be linked to a substrate via a pair of coupling molecules, such as a streptavidin/biotin pair or carboxyl binding group. Include option of linkage to substrate via a linker or pair of mutually binding molecules.

### Performance Parameters

Methods, reagents and kits of the invention preferably meet one or both of the following performance parameters:
1, sufficient sensitivity to detect bacterial DNA from human whole blood, wherein the bacterial DNA is present in bacteria in the blood at levels down to 100, 10, 5 or 1 CFU per ml of whole blood.
2, sufficient binding specificity to bind E. coli DNA in vitro when in a mix with human DNA, such that at least 95% or at least 99% of total DNA binding is with the E. coli DNA, when less than 0.01 ng of the E. coli DNA and 10 ng of the human DNA are present in an aqueous solution of 1 ml in volume.

### Rinsing

Methods of the invention may include subsequent to binding of a protein comprising the TgMcrBΔ185 N-terminal domain of Thermococcus gammatolerans McrB protein or a derivative thereof to a DNA of bacterial origin, on more rinsing steps whereby the substrate is rinsed with one or more rinsing solutions in order to wash away compounds (including but not limited to DNA of eukaryotic origin non-specifically bound or only weakly bound to the substrate) which is trapped in or on the substrate, whilst selectively retaining any DNA of bacterial origin. Kits of the invention may optionally include one or more rinse solution. An exemplary rinse solution consists of 10 mM Tris, 500 mM NaCl, 0.1% Tween20, 10 mM CaCl₂.

### Elution

Following selectively binding DNA of bacterial origin with a protein comprising the TgMcrBΔ185 N-terminal domain of Thermococcus gammatolerans McrB protein or a derivative thereof which has been immobilised into a substrate, it may be desired to release (“elute”) the DNA of bacterial origin from the substrate. Methods of the invention therefore optionally further comprise an elution step. Elution may be achieved by disrupting the binding interactions between binding DNA of bacterial origin and the protein comprising the TgMcrBΔ185 N-terminal domain of Thermococcus gammatolerans McrB protein or a derivative thereof. That may be achieved by altering subjecting the bound DNA of bacterial origin to an elution solution. An elution solution may be formulated to have a temperature, ionic strength or composition suitable for disrupting binding. An exemplary rinsing solution is a solution comprising 5M guanidine thiocyanate.

According to certain optional embodiments, elution of the DNA of bacterial origin may be carried out by cleavage of the linker binding the protein comprising the TgMcrBΔ185 N-terminal domain of Thermococcus gammatolerans McrB protein or a derivative thereof to the substrate. The linker may be a chemically cleavable linker or photo-cleavable linker. Kits of the invention may optionally include one or more elution solution, and optionally one or more reagent or piece of equipment for carrying out linker cleavage.

### Additional Steps

A method of the invention may optionally include a step of subjecting the DNA of bacterial origin to genetic sequencing (especially next generation sequencing). Because the invention provides means of selectively enriching for DNA of bacterial origin, in a non-sequence specific fashion, it is especially suitable for use with methods whereby the eluted DNA of bacterial origin is sequenced (in whole or part) or subjected to other molecular analysis such as polymerase chain reaction based approaches. The method is also suitable for integration into a method whereby the DNA of bacterial origin is cloned, bound to a nucleic acid microarray or amplified (for example by PCR). Methods of the invention encompass rather optional steps of analysis (for example computational analysis) of the results. Methods of the invention may optionally be carried out in order to detect or identify bacterial species and/or bacterial strains (including novel species and/or strains), to detect mutations, to identify the presence of alleles. In certain preferred embodiments, methods of the invention are carried out in order to detect the presence of genetic sequences which cause or are likely to cause genetic resistance in the bacteria to antimicrobial agents.

Kits of the invention may optionally comprise further components for carrying out one of more of the additional steps specified herein. Kits of the invention may optionally comprise of instructions and/or software for directing a user, guiding a user or assisting a user in carrying out a method of the invention, optionally including instructions and/or software for directing a user, guiding a user or assisting a user in carrying out one or more additional steps.

Various aspects of the invention are illustrated below by means of non-limiting examples.

### EXAMPLES

### Brief Experimental Methods

### Bacterial Strains and Growth

Escherichia coli BL21 (DE3), E. coli K-12 and Staphylococcus aureus ATCC 29213 were cultured in Luria broth, Terrific broth or Tryptic Soy broth/agar at 37° C. overnight as required. When required bacterial DNA was extracted using Qiagen DNeasy blood and tissue kit, with lysostaphin and lysozyme addition to the lysis buffer to ensure effective lysis of S. aureus.

### TgMcrBΔ185 Expression and Purification

For in vitro assays using TgMcrBΔ185 we designed plasmid backbone based on the pET system and cloned in the N-terminal domain of T. gammatolerans TgMcrBΔ185 with a N-terminal His tag, using Golden Gate cloning methodology. Successful cloning was confirmed with PCR and sequencing and the protein was expressed overnight in Terrific broth in BL21 (DE3). The TgMcrBΔ185NHis6 protein was then lysed and purified using the BioRad NGC chromatography system and buffers comparable to those used in Hosford et al. 2020, and buffer exchanged into PBS prior to use in subsequent assays. When required TgMcrBΔ185 was biotinylated using biotin-DPEG4-TFP or biotin-PEG12-TFP esters, at a 20 molar excess, prior to purification using Zeba columns and use in magnetic bead based assays.

### ELISA Assay

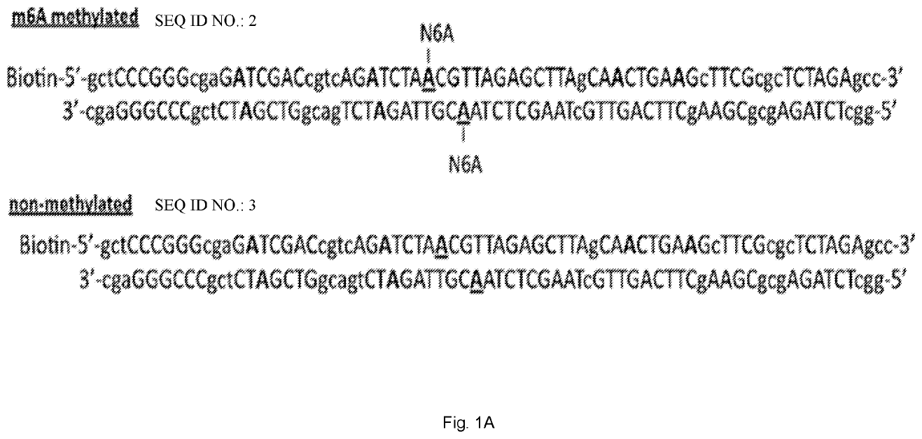

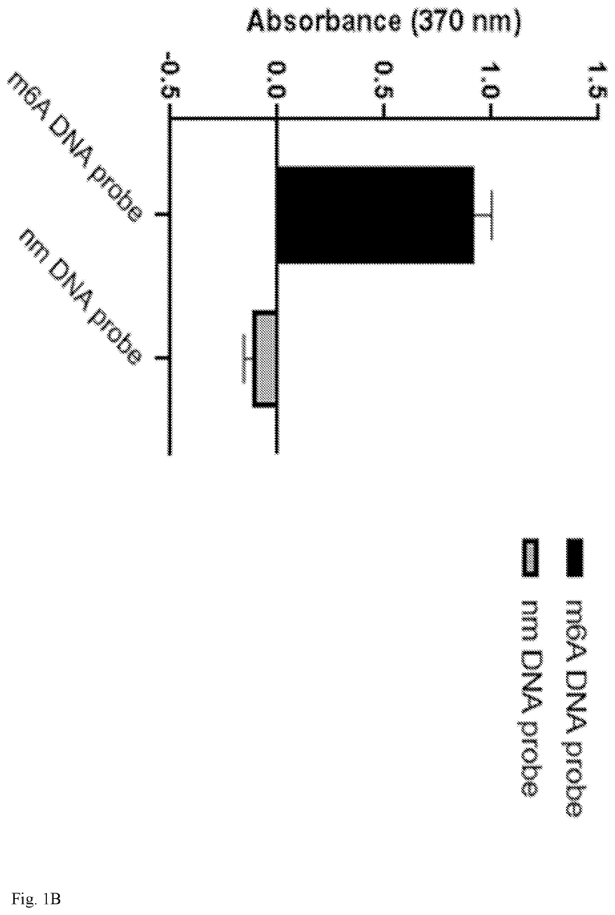

An ELISA based assay was developed in-house to determine TgMcrBΔ185NHis6 specificity of binding to methylated DNA probes. 2 nM of biotinylated methylated or unmethylated DNA probes (see FIG. 1 for sequences) were bound to ThermoFisher streptavidin coated 96 well ELISA plates, prior to washing, blocking and addition of purified TgMcrBΔ185NHis6. Following an additional wash step binding of our bound TgMcrBΔ185 to the DNA probes was detected using Novagens anti-His-HRP conjugate antibody and Pierce 1-step Ultra TMB-substrate.

### Magnetic Bead Assay

Bead-based studies have largely been performed using streptavidin MyOne C1 Dynabeads for binding to biotinylated TgMcrBΔ185 to 10 ng of bacterial (E. coli K-12 or S. aureus ATCC 29213) or human genomic DNA. Further assays have been performed using 100 ng E. coli or human DNA or a mix of 1 μg of human DNA and 1 ng E. coli genomic DNA. The assay was performed by first binding 10 ng/μL of biotinylated to 20 μL of MyOne C1 Dynabeads for 30 minutes with mixing end-over-end. Following protein binding the beads were then washed twice prior to addition of the DNA and a further 5-30 minute incubation with end-over-end mixing at room temperature. Following DNA binding the supernatant was removed and the beads washed twice and the DNA was eluted off the beads using 5 M guanidine thiocyanate and the sample was dialysed against deionised water prior to use in qPCR analysis as described below.

qPCR

Singleplex quantitative qPCR was performed using TaqMan Advanced Fast master mix and primer/probes designed against bacterial 16S, either E. coli or S. aureus (with an incorporated FAM probe) or mammalian 18S (HEK/VIC probe). Relative DNA concentrations were determined against a standard curve performed on each qPCR plate, against either bacterial or human DNA. qPCR was performed with at least two technical replicates, using the QuantStudio 5.

### Results

As shown in FIG. 1, purified TgMcrBΔ185NHis6 shows specific binding to m6A methylated DNA probes in an ELISA based assay. FIG. 1A shows methylated and non-methylated dsDNA probes that were designed based on consensus sequences known to show m6A methylation in a range of bacterial species. The one m6A site included was designed based on the DNA probe used in Hosford et al. (2020) paper sequence. Other sites have been included to allow for assay flexibility for example, CTGAm6AG is commonly found in Enterococcus faecalis and GATCGVNY which is associated with m6A methylation is found in Staphylococcus aureus

FIG. 1B shows ELISA data results showing specific purified TgMcrBΔ185NHis6 binding to the methylated DNA probe only. The ELISA was performed by binding the biotinylated m6A methylated (black) or non-methylated (nm=grey) DNA probes to streptavidin coated plates, followed by blocking and overlaying with TgMcrBΔ185NHis6 and detection of protein binding to the DNA probes following addition of anti-HisHRP. Absorbance was measured at 370 nm following addition of the TMB ELISA substrate. Results show the data normalised to the no DNA control that is the average of two experiments, each with at least 2 technical replicates.

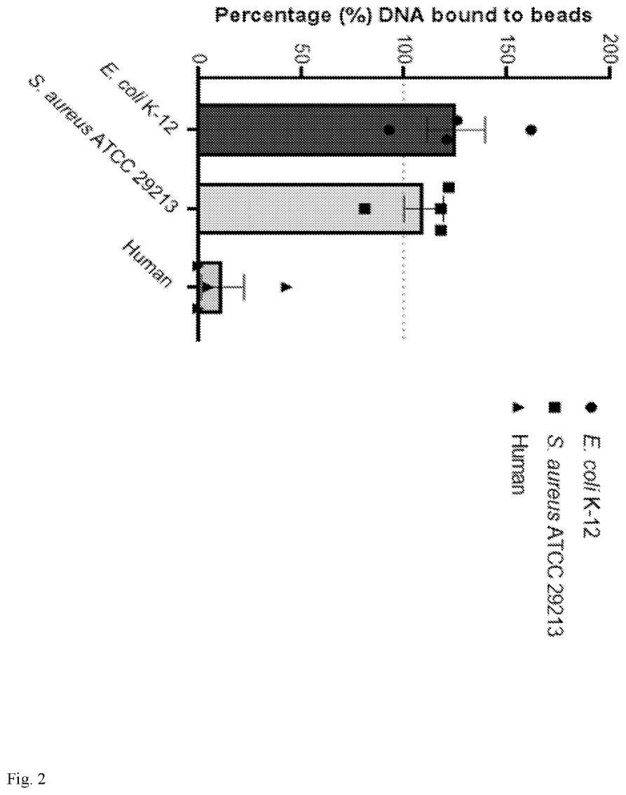

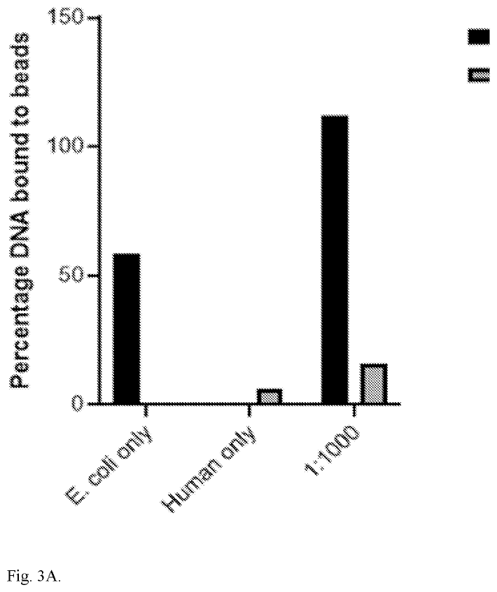

FIG. 2 shows percentage genomic DNA bound to biotinylated TgMcrBΔ185 bound to streptavidin coated magnetic beads. Data shows the average percentage (+/−SEM) of genomic DNA bound and eluted from Dynabeads MyOne C1 streptavidin magnetic beads coated with biotinylated TgMcrBΔ185, based on a 10 ng total input amount. Results show the average and technical variation of two experiments conducted with two technical replicates. The data shows 100% recovery of 10 ng E. coli and S. aureus genomic DNA when, and variable but low levels<40% recovery of equivalent concentrations of human DNA.

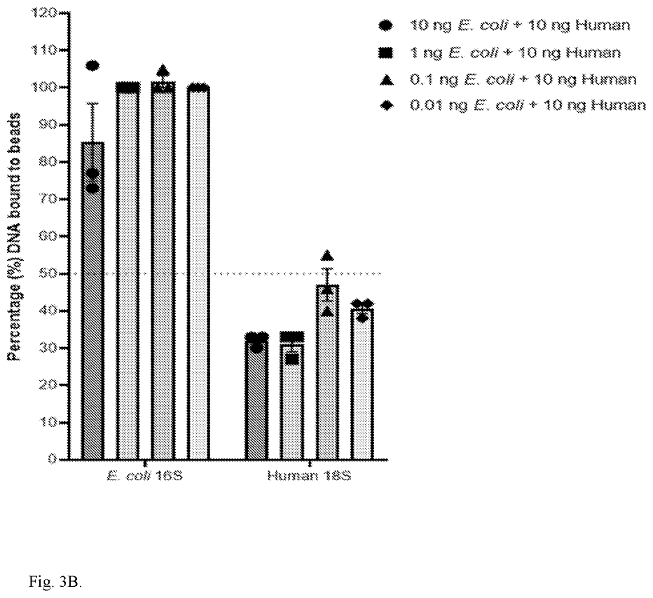

FIG. 3 shows percentage E. coli and human DNA bound to biotinylated TgMcrBΔ185 coated magnetic beads. FIG. 3A data shows results of one technical replicate for DNA binding to beads. The assay was performed with either E. coli or human DNA alone at 100 ng, or a mix of E. coli and human DNA at a 1:1000 ratio, using 1 ng E. coli and 1 μg human DNA respectively. This initial experiment shows the utility of the invention for enrichment of bacterial DNA from mixed samples. FIG. 3B data shows results from three technical replicates showing 85-100% recovery of E. coli DNA (at 10 ng, 1 ng, 0.1 ng and 0.01 ng) when mixed with 10 ng human genomic DNA. The results show that TgMcrBΔ185 protein preferentially binds to bacterial DNA when mixed with human genomic DNA which shows between 30 to 45% binding to the beads.

1. A method of selectively binding DNA of bacterial origin comprising the step of:
i) contacting a sample solution containing DNA of bacterial origin with a protein comprising the TgMcrBΔ185N-terminal domain of Thermococcus gammatolerans McrB protein or a derivative thereof which has been immobilised into a substrate whenever the solution further contains DNA of eukaryotic origin at a concentration of more than 1-100 times that of the DNA of bacterial origin

2. A method according to claim 1 wherein the substrate is a magnetic substrate, for example, a magnetic bead.

3. A method according to claim 1 having the following additional subsequent step:
ii) rinsing the substrate with a rinse solution to wash off any DNA of eukaryotic origin non-specifically bound or only weakly bound to the substrate whilst selectively retaining any DNA of bacterial origin.

4. A method according to claim 3 having the following additional subsequent step:
iii) eluting the DNA of bacterial origin from the substrate.

5. A method according to claim 4 having the following additional subsequent step:
iv) subjecting the eluted DNA of bacterial origin to a further analytical process, for example, quantification Polymerase Chain Reaction (PCR), genetic cloning or sequencing.

6. A method according of claim 1 which is a method for identification of the presence of one or more bacterial species in the sample solution which preferably is or is derived from a clinical sample previously taken from a patient.

7. A method according of claim 1 comprising an additional proceeding step of obtaining the sample solution from a clinical subject or deriving the sample solution from a clinical sample obtaining from a clinical subject.

8. A method according of claim 1 which is a method for the rapid diagnosis of sepsis or blood stream infections

9. A method according of claim 1 which is a method for the rapid identification of antimicrobial resistance in bacteria contributing to the DNA of bacterial origin.

10. A method in claim 8 which is a method of treating a patient having or suspected or having a bacterial infection such as sepsis or a blood stream infection, the method comprising the additional subsequent steps of:
A—selecting a suitable antimicrobial agent for treating the bacterial infection of the patient by using the diagnostic or antimicrobial resistance information to choose an appropriate antimicrobial agent; and
B—administering said appropriate antimicrobial agent to the patient.

11. A reagent comprising:
a protein comprising the TgMcrBΔ185N-terminal domain of Thermococcus gammatolerans or a derivative thereof which has been immobilised into a substrate and which is capable of binding DNA of bacterial origin in preference to DNA of eukaryotic origin.

12. A reagent according to claim 11 wherein the substrate is a magnetic substrate, for example a magnetic bead.

13. A reagent according to claim 11 wherein the substrate is a nucleic acid array.

14. A kit comprising a reagent according of claim 11 together with one or more further components selected from:
1. a wash solution for washing the substrate of any DNA of eukaryotic origin which may have bound non-specifically or weakly to the substrate whilst selectively retaining any DNA of bacterial origin, and
2. an elution solution for eluting DNA of bacterial origin from the substrate.


SEQUENCE LISTING (3 sequences)

SEQ ID NO: 1 - protein, 185 aa, Thermococcus gammatolerans
MENQLFIIGIGTGTDEYENFEETILKGVKRNELEGQIGPDILDNCCSDVCYFWGRSKETIYEKKIDKGDMVLFYVGKRISRNKVDLNQETAVYLGIICETVEISENDVSFLNDFWRKGENFRFLMFFKKKPEKLHHSINEINSKLGYNPDYFPIAGYVKPERMSGVYDILKNILKKRGILKESDS

SEQ ID NO: 2 - DNA, 140 bp, synthetic construct
  source 1..140: mol_type = other DNA
  modified_base 31: mod_base = m6a
  modified_base 104: mod_base = m6a
gctcccgggcgagatcgaccgtcagatctaacgttagagcttagcaactgaagcttcgcgctctagagcccgagggcccgctctagctggcagtctagattgcaatctcgaatcgttgacttcgaagcgcgagatctcgg

SEQ ID NO: 3 - DNA, 140 bp, synthetic construct; identical to SEQ ID NO: 2
  source 1..140: mol_type = other DNA